(19) United States
(12) Patent Application Publication
(10) Pub. No.: US 2025/0118396 A1
(43) Pub. Date: Apr. 10, 2025

(54) METHOD AND DEVICE FOR PROCESSING EXPERIMENTAL DATA BY MACHINE LEARNING

(71) Applicant: COMMISSARIAT A L'ENERGIE ATOMIQUE ET AUX ENERGIES ALTERNATIVES, Paris (FR)

(72) Inventors: Mihai Cosmin MARINICA, Gometz le Châtel (FR); Alexandra GORYAEVA, Rouen (FR); Clovis LAPOINTE, Reims (FR); Wesley UNN TOC, Toulouse (FR); Jean-Luc BECHADE, Dourdan (FR)

(73) Assignee: COMMISSARIAT A L'ENERGIE ATOMIQUE ET AUX ENERGIES ALTERNATIVES, Paris (FR)

(21) Appl. No.: 18/836,145

(22) PCT Filed: Feb. 9, 2023
(86) PCT No.: PCT/FR2023/050179

§ 371 Date: Aug. 6, 2024

(30) Foreign Application Priority Data
Feb. 9, 2022 (FR) FR2201130

(51) Int. Cl. G16C 20/70; G16C 20/20

(52) U.S. Cl. CPC G16C 20/70; G16C 20/20

(57) ABSTRACT

A computer-implemented method for processing experimental data of a solid to be characterized, including atoms and including one or more defects, the experimental data coming from at least one sensor and having a multimodal distribution. The method includes the representation, in a space called descriptor space, of dimension K, comprised between 10 and 10⁸, of one or more reference solid(s) and the data; the calculation for at least one portion of the atoms of the solid to be characterized of an experimental confidence score in the descriptor space, relative to the atoms of the reference solid; and the classification of the atoms of the structure depending on the experimental confidence score.

### TECHNICAL FIELD AND STATE OF PRIOR ART

The invention relates to the field of processing experimental data resulting from a measurement on a solid.

In the case of measurements carried out with Tomographic Atom Probe (TAP) type technique or by X-ray diffraction with Synchrotron Radiation (RS) or by Transmission Electron Microscopy (TEM), there is no technique allowing effectively analysing the data which result from the measurements. These measurements reproduce massive amounts of data, for which few analysis tools are available. The known tools do not allow these data to be used in a reproducible, non-biased manner and without human interpretation error.

A first problem is therefore that of using such data in a reproducible, unbiased manner and without human interpretation error.

According to another aspect, recent publications, and in particular the article by Goryaeva et al., Reinforcing materials modeling by encoding the structures of defects in crystalline solids into distortion scores, Nature Communications, https://doi.org/10.1038/s41467-020-18282-2 (2020), exclusively propose analysis solutions for data from simulations at the atomic scale, i.e. devoid of any uncertainty specific to experimental data. In addition, as indicated in this document, the method which is proposed therein is used only with data distributed in a unimodal manner. However, data resulting from the experiment never satisfy this unimodal character, but are always multimodal, for reasons inherent, in particular, to the various sources of noise that the experiment involves. Moreover, the interpretation of data from experiments at the atomic scale has never before been the subject of an analysis.

FIG. 4 represents analysis results obtained by implementing the technique described in the article by Goryaeva et al. mentioned above, by implementing the distortion score of this document for a perfect solid, then for a solid including 30% missing atoms, and finally for a solid including 50% missing atoms (50% missing atoms is a situation frequently encountered in TAP-type experiments). It can be seen that the distributions obtained for this distortion score are very different from each other: thus, 100% of the results obtained for solids including many defects are considered as errors or outliers. In other words, if the technique described in this document gives satisfactory results for a perfect theoretical solid, it is not applicable to real solids, which may have significant defects and for experimental data distributed according to multimodal distributions. In other words, real data is too noisy and much more complex than “in silico” data, which, by comparison, is simple, Gaussian and clean; for the latter, the teaching of the prior art is suitable, but it is not suitable for real solid data, which is very noisy and disordered.

There therefore also arises the problem of processing and/or interpreting experimental data with an atomistic resolution and the identification and/or the classification of defects.

There is also the problem of processing and/or interpreting experimental data with a very reduced or reduced or acceptable speed relative to the known techniques.

### DISCLOSURE OF THE INVENTION

The invention aims at solving one or more of these problems.

It proposes a method adapted for analysing experimental data as well as their interpretation based on the characteristics specific to each type of experiment.

The invention firstly relates to a method for processing data, preferably implemented by a computer, coming from at least one sensor used in the context of an experiment for characterising a solid, this solid consisting of atoms and including one or more defect(s), this method including:
the calculation and/or the representation, in a space called descriptor space, of dimension K>3 or even K>>3, for example comprised between, on the one hand, 10 or 50 and, on the other hand, 10³ or 10⁴, or even 10⁸, of one or more reference solid(s) and said data;
the calculation, preferably in the descriptor space, of an experimental confidence score of each atom of said solid to be characterised, relative to at least the atoms of said structure(s) or of said reference solid;
the grouping and/or the classification of the experimental data and/or the atoms of the structure depending on the experimental confidence score; thus, it is possible for example to identify the atoms which are grouped into clusters of defects in the sample of material or solid which is analysed.

Such data, in particular the noise that affects them, has a multimodal distribution, unlike the unimodal data which is treated in the paper by Goryaeva et al. already commented on above.

This type of multimodal distribution is typical of data from experimental analyses, in particular data obtained by techniques such as Tomographic Atom Probe (TAP) or even by or by Synchrotron Radiation (SR) X-ray diffraction or by Transmission Electron Microscopy (TEM).

The invention implements the representation and analysis of experimental data, usually done in the real 3D space, in an increased K-dimensional space, from a few tens to thousands of dimensions, for example between, on the one hand, 10 or 50 and, on the other hand, 10³ or 10 or even 10⁸. This new representation of the data is ensured by one or more mathematical function(s) called “the descriptor functions”.

The invention allows processing and/or interpreting experimental data with an atomistic resolution and/or identifying and/or classifying defects which are or may possibly be present in the studied material or solid.

According to one aspect, the invention implements an interpretation and/or an analysis of the data from experiments, at the atomic scale, in the abstract descriptor space.

The solid or material which is studied and/or to be characterised can be previously subjected to an experimental technique which allows accessing, thanks to a resolution at the atomic scale, to the arrangements of the atoms which may (or not) include one or more defect(s). The experimental data can for example be obtained by Tomographic Atom Probe (TAP) technique or by Transmission Electron Microscopy (TEM) or by X-ray diffraction, for example from Synchrotron Radiation.

The invention allows accelerating the analysis of data from experiments, at the atomic scale; it also allows data to be used in a reproducible, non-biased manner and without human interpretation error.

A method according to the invention can be preceded:
a step of processing or preprocessing and/or preparing experimental data, preferably taking into account experimental particularities: noise/detection uncertainty etc;
and/or a step of calculating or forming a descriptor space, and/or one or more descriptor function(s), for example depending at least on the distances between the atoms and/or the angles between the directions connecting each atom of the solid or the sample which is studied to its different neighbours in its network. The atomic descriptors, of dimension K>3 or even K>>3, for example between, on the one hand, 10 or 50 and, on the other hand, 10³ or 10⁴, or even 10⁸, preferably preserve all or part of the symmetries and/or the chemical nature of the atomic structure(s) resulting from the experiment and/or used for reference.

For example, after the acquisition of data made by an experimental technique allowing analysing the matter at the atomic scale (for example here the TAP, but also TEM or XRD with synchrotron radiation) the invention allows transforming this data from the real 3D space to a higher dimension K space (see indications above concerning K) of said descriptor space using the mathematical functions of descriptor. A descriptor function allows increasing the dimensionality K of the data representation space while preserving the symmetry of atomic arrangement inherent to the used experimental analysis technique, for example TAP.

In a method according to the invention the representation step is preferably carried out using a descriptor (of the so-called “FastGraph” type) which implements, for each atom j, a graph Gj whose nodes are neighbours, more or less close, to the atom j, this graph then being pixelated in the form of a matrix; the graph is preferably a dense, non-directional graph, the nodes or vertices of the graph being for example, or corresponding to, the very atoms of the atomic environment of a central atom and for example with edges with weight weighted by the interatomic distances. The matrix is preferably the pixel matrix of such a dense, non-directional graph. A method according to the invention may include a step of distributing or grouping the atoms detected by class of defects, by a convolutional neural network (CNN) type method.

A method according to the invention can be preceded:
the selection of a cut-off radius R_{c}, which defines the environment of each atom j (including all atoms present in the close neighbourhood of the atom j and which are included in the cut-off radius R_{c});
and/or the definition of one or more symmetry (symmetries) at least in the volume of radius Rᵢ around each atom.

In a method according to the invention, the set or the class of reference structures may include “perfect” solid environments and/or well-predefined structures, which can include point defects (dp) and/or clusters of point defects and/or extensive defects such as dislocation lines.

A method according to the invention can implement a comparison, for example in the descriptor space, between one or more reference structures and the structure of interest (on which a study or analysis is carried out, for example in TAP). This comparison can be made by calculations of the statistical distances, for example in the descriptor space. This statistical distance, which measures the difference between the descriptor(s) of the reference structure(s) and the descriptor of the structure of interest, will then allow identifying the defect(s) of the structure of interest. It is therefore possible to locate the structures of the defects (“unusual” structures or “anomalies”) by eliminations of the environments close to the reference structures (if the reference is represented by the perfect solid, without defects).

Advantageously, in the descriptor space, it is possible to compare the experimental structure or structure of interest (object of the analysis which is carried out) relative to one or more reference structure(s), which can for example come from data acquired during an experimental measurement on a defect-free sample (which can be similar to a perfect solid).

This comparison can be made objective, reproducible, unbiased and without human interpretation error by using the notion of statistical distance associated with each atom which constitutes the two classes of structure. In statistical mathematics, the notion of statistical distance is used to measure the difference between two probability distributions: within the scope of the present invention, the distributions of the atomic descriptors, on the one hand, of the reference and, on the other hand, of the experiment are compared.

Therefore this comparison can be made by calculating the statistical distances, also called experimental confidence score, between the two classes of experimental and reference structures. This distance is ambiguous and mathematically difficult to define in the real 3D space. In contrast, in the increased K-dimensional space (the descriptor space), this distance is less ambiguous and more mathematically robust. This statistical distance measures the difference between the descriptor(s) of the atoms which constitute the reference structure(s) and the atomic descriptor(s) of the structure of interest analysed by the experiment.

Thanks to this experimental confidence score, it is therefore possible to locate the atoms which are part of defect structures (“unusual” structures or “anomalies”), for example by eliminating environments close to the reference structures (if the reference is represented by the perfect solid, without defect). Using for example the experimental confidence score resemblance of the “unusual” atoms, it is then possible to group the similar atoms and classify each grouping or cluster. As indicated above, the descriptors can take into account the distances between neighbouring atoms in the network of the reference solid and/or the angles between the directions which connect an atom to its different neighbours in this network, in particular in the cases where the experimental data is obtained by Tomographic Atom Probe (TAP) technique.

A method according to the invention may further include a step of learning a method for calculating, for example a statistical distance, of said experimental confidence score. Such a step can implement a machine learning or deep learning method or even an anomaly detection or novelty detection method, for example a physical statistical distance calculation with an SVM type technique or a neural network.

The classification of the atoms can itself implement a classification algorithm of the DBScan or neural network or SVM, or MCD or any other “clustering” method type.

A method according to the invention may further include a step of distributing or grouping the atoms detected by class of defects, by a machine learning or deep learning type multimodal method or a clustering and classification method such as DBSCAN or a “Gaussian Mixtures” or neural network type method.

The invention also relates to a device for processing experimental data from solids to be characterised, consisting of atoms and including one or more defects, this device implementing, or being adapted or programmed for, a method as described above or in the present application.

The invention also relates to a device for processing experimental data from a solid to be characterised, consisting of atoms and including one or more defects, this device including:
means adapted and/or programmed to represent, in a space called descriptor space, of dimension K>>3, at least one reference solid or data from this reference solid, and said data,
means adapted and/or programmed to calculate an experimental confidence score, in the descriptor space, for example as defined above or in the remainder of this application, for at least one portion of the atoms of said solid to be characterised, relative to the atoms of said reference solid;
means adapted and/or programmed to classify atoms of a solid and/or data to be analysed from this solid, depending on said experimental confidence score.

A device according to the invention can further include means adapted and/or programmed to:
form or calculate a descriptor space from experimental data, for example from data of at least one reference sample depending on at least the distances between the atoms and the angles between the directions connecting the atoms of this solid;
and/or form or calculate a descriptor space, and/or one or more descriptor function(s), for example depending on at least the distances between the atoms and/or the angles between the directions connecting each atom of the studied solid or sample to its different neighbours in the network of the solid to be characterised or studied; the atomic descriptor(s) may have the properties already explained above and/or detailed subsequently in the present application;
and/or to implement a step of processing or preprocessing and/or preparing experimental data, preferably taking into account the experimental particularities: noise/detection uncertainty etc.;
and/or implement a step of learning a method for calculating, for example a statistical distance, of said experimental confidence score; for example such a step can implement a machine learning or deep learning method or even an anomaly detection or novelty detection method, for example a statistical distance calculation or else an MCD or Mahalanobis type method (if the multimodal nature of the experimental data is well established and known and usable) or else a calculation of physical statistical distance adapted to the multimodal experimental data by an SVM type technique or a neural network;
and/or, for the classification of the atoms, implement a multimodal classification algorithm of the DBScan or neural network or SVM, or MCD or any other “clustering” method type;
and/or a distribution or a grouping of the atoms detected by class of defects, by a machine learning or deep learning type method or a clustering and classification method such as DBSCAN or a “Gaussian Mixtures” or neural network type method;
select a cut-off radius R_{c}, which defines the environment of each atom (including all atoms present in the close neighbourhood included in the cut-off radius R_{c});
and/or enter or select one or more symmetries, at least in a volume of radius Rᵢ around each atom.

A device according to the invention may include means adapted and/or programmed to implement a representation step using a descriptor (of the so-called “FastGraph” type) for which, for each atom j, a graph Gj whose nodes are neighbours, more or less close, to the atom j, this graph then being pixelated in the form of a matrix, preferably a matrix of pixels of a dense, non-directional graph, the nodes or vertices of the graph being for example the, or corresponding to, the very atoms of the atomic environment of a central atom and for example with edges with weight weighted by the interatomic distances.

A device according to the invention may include means adapted and/or programmed to implement a machine learning or deep learning method or even an anomaly detection or novelty detection method, by a convolutional neural network.

In a method or a device according to the invention, the implementation of a “FastGraph” type descriptor (based on graphs and matrices) allows the rapid learning by a convolutional neural network (CNN) of the experimental multimodal noise.

By using this “FastGraph” descriptor, the noise from the experiment (including the missing atoms) is perceived by the CNN network as contrast variations on the matrix elements M of the atomic neighbourhood. Each element of the matrix becomes a pixel of an image, and is therefore usable by the CNN network.

A device according to the invention can be connected to a detector, for example a detector of a Tomographic Atom Probe (TAP) system or an X-ray detector associated with a Transmission Electron Microscopy (TEM) system or an X-ray diffraction system, for example from Synchrotron radiation.

### BRIEF DESCRIPTION OF THE DRAWINGS

FIG. 1 represents steps of a method according to the invention.

FIGS. 2a-2d represent aspects of a “FastGraph” type descriptor.

FIG. 3 represents results obtained with a “FastGraph” type descriptor coupled with a convolutional neural network.

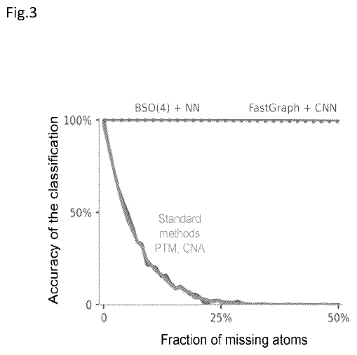

FIG. 4 represents results obtained with a method of the prior art.

FIGS. 5 and FIG. 6 represent data acquisition and processing means which can be used within the scope of the present invention.

### DETAILED DESCRIPTION OF PARTICULAR EMBODIMENTS

The invention will first be explained in connection with a specific technique for analysing a material, namely the tomographic atom probe (TAP) technique. This technique is for example described in the work “Atom probe Tomography”, ISBN 978-0-12-804647-0, Editors Williams Lefebvre-Ulrikson, François Vurpillot, Xavier Sauvage, Academic Press, 2016. But the invention can be applied to other techniques for analysing a material, for example to the analysis of images in transmission electron microscopy or to an X-ray analysis technique, for example by diffraction, the X-radiation possibly coming from Synchrotron radiation.

In all these techniques, and in many others, the obtained experimental data are never with unimodal distribution, but are always multimodal, for reasons inherent, in particular, to the various sources of noise that each experiment involves.

The analysed solid material includes a crystalline structure consisting of atoms disposed in a network. This network may include defects that need to be identified and/or characterised. Any two neighbouring atoms in this network are separated by an interatomic distance, and the directions connecting an atom to each of its neighbours are separated by different angles; see for example:
https://diamantgraphite.wixsite.com/tpecristaux/systmes-cristallins

In addition, real samples can present defects in a significant proportion, for example up to 30 or 50% missing atoms as already mentioned above, which reinforces the very more complex nature of the obtained data, which are very far from the theoretical data used in the context of the article by Gorayeva et al. already mentioned above.

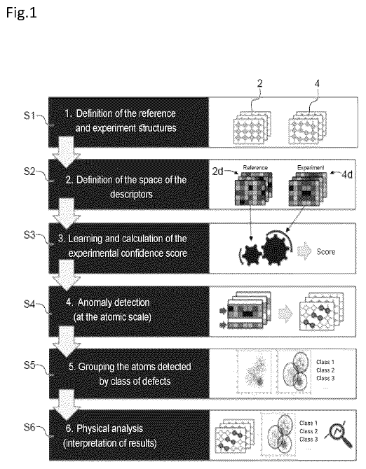

Steps of an exemplary embodiment of a method according to the invention are illustrated in FIG. 1.

In a first step (S1) one or more structure(s) or solid(s) to be analysed and one or more reference structure(s) 2 are defined.

The reference structures 2 can be of different types. They can for example come from data acquired during an experimental measurement (same experimental method as the method to obtain the structure 4) on a defect-free sample.

This step S1 can therefore be preceded by an experimental measurement step, by TAP technique in the considered example, generating data from at least one structure to be analysed and/or data from a defect-free sample.

In addition, at least one portion of the reference structure(s) 2 may also be derived from in silico data obtained by numerical simulation. One portion of this in silico data can be generated by numerical simulations taking into account the particularity of the experiments.

In the example of the tomographic atom probe (TAP) technique, these digital data take into account:
specific characteristics of this analysis technique, for example the spatial and chemical uncertainties; these parameters are inherent to the experimental technique as described in the work “Atom probe Tomography” which is already mentioned above;
and/or the different behaviours of the atoms during their evaporation (related to the TAP technique), such as the crystallographic direction and/or the presence of another phase (clusters, or defects . . . ) within the sample to be analysed. These parameters are also inherent to the experimental technique and are described in the work “Atom probe Tomography” (mentioned above).

In a second step (S2) a descriptor space is defined, which is a unique mathematical space for the representation of the experiment data 4 and the reference 2 named below 2_{d} and 4_{d} respectively. In particular, each atom can be defined by its geometric environment with all atoms present in the close neighbourhood included within a certain cut-off radius R_{c}. This neighbourhood of the atom i can be completely described by the positions of Nᵢ neighbours: the set of Nᵢ vectors

$${R}_{i} = ( {r}_{{i}_{1}} , {r}_{{i}_{2}} , … , {r}_{{i}_{{N}_{i}}} ) \qquad [ Math 1 ]$$

where each rᵢ_{ₖ} is a 3-dimensional vector representing Cartesian coordinates of k^{th} neighbour of the i^{th} atom: xᵢ_{ₖ}, yᵢ_{ₖ}, zᵢ_{ₖ}

An atomic descriptor function Dᵢ can transform and project the environment

$${R}_{i} = ( {r}_{{i}_{1}} , {r}_{{i}_{2}} , ... , {r}_{{i}_{{N}_{i}}} ) \qquad [ Math 2 ]$$

in a K-dimensional space (see the indications above regarding the value of K). These functions Dᵢ(Rᵢ) can take into account all symmetries of the neighbourhood Rᵢ or at least one or more thereof.

Preferably, the mathematical functions of the descriptors preserve the topology of the experimental atomistic data by keeping the physical symmetry (symmetries) associated with the crystal structure of the material, for example the rotations, and/or the translations and/or the permutations of the atoms.

This descriptor space, which is a Euclidean mathematical space, is preferably of dimension K much larger than 3 (3 is the dimension of the real space of data 2 and 4); it can for example be generated by applying one or more descriptor functions to each atom from experimental 4 and reference 2 data. In other words, each atom of a sample 2 and 4 can be described using its representation in the descriptor space i.e. of a vector in a K-dimensional space, K>3 or even K>>3, for example K comprised between, on the one hand, 10 or 50 and, on the other hand, 10³ or 10⁴ or even 10⁸. (10 or 50≤K≤10³, 10⁴ or 10⁸).

The descriptor functions preferably preserve the geometric (including crystallography) and chemical symmetries of the solid (i.e. the invariance to the permutation of the atoms of the same chemical species), for example by taking into account the coordinates of the atoms in the network and/or or the distances between neighbouring atoms in the network and/or the angles between the directions which connect an atom to its different neighbours in the network of the solid and/or the structural symmetries of the material or the solid and/or the density (densities) of atoms in the network.

Examples of descriptors which use the distances and/or the angles between the atoms are given in J. Behler et al., Phys. Rev. Lett. 98, 146401 (2007).

Examples of descriptors that use the spectral analysis of the atomic densities are given in the thesis of A. P. Bartok “Gaussian Approximation Potential: an interatomic potential derived from first principles Quantum Mechanics”, Ph.D. Thesis, University of Cambridge (2009) or in the article by A. P. Bartok et al., Phys. Rev. B 87, 184115 (2013), or in the article by M. Eickenberg et al., in Advances in Neural Information Processing Systems 30, edited by I. Guyon, U. V. Luxburg, S. Bengio, H. Wallach, R. Fergus, S. Vishwanathan, and R. Garnett (Curran Associates, Inc., 2017) p. 65406549.

Examples of descriptors that use a tensor description of atomic coordinates are given in the article by A. Shapeev, Multiscale Model. Sim. 14, 1153 (2016) or in the article by E. V. Podryabinkin et al., Comput. Mater.Sci.140, 171 (2017). Examples of descriptors that preserve the symmetry relative to the rotations and permutations are given in C.v.d.Oord et al., Machine Learning: Science and Technology 1, 015004 (2020), Y. Lysogorskiy et. al. npj Computational Materials volume 7, article 97 (2021).

In order to best process the complex experimental data, a special class of descriptor (named “FastGraph”) is used. This class of descriptor allows carrying out a rapid assessment of a high-dimensional system of, for example, 106 to 109 atoms, in particular the results of tomographic atom probe (TAP) experiments, with the limited computational capacity of a usual office computer.

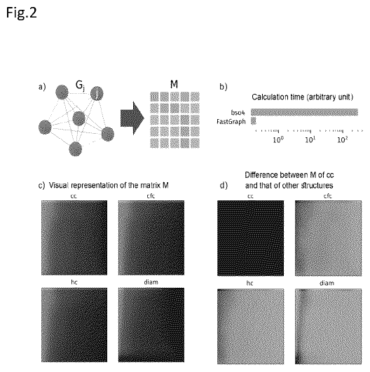

This type of descriptor will first be explained in connection with FIGS. 2a-2d. FIG. 2a represents the local neighbourhood of a central atom j, in the form of a graph Gⱼ, which is then encoded in the form of a pixelated matrix M. This method is efficient from the numerical point of view and allows, as illustrated in FIG. 2b, an acceleration of up to 10,000 in the calculation speed, in comparison with a BSO(4) spectral descriptor used in the article which is already commented above by Gorayeva et al. In addition, the design of a descriptor of the FastGraph class allows the rapid learning by the convolutional neural network (CNN) of experimental multimodal noise. By using a FastGraph descriptor, the noise from the experiment (including the missing atoms) is perceived by the CNN networks as contrast variations on the matrix elements M of the atomic neighbourhood. Each element of the matrix becomes a pixel of an image, therefore easily usable by the CNN network. See also Neural Networks and Deep Learning: A Textbook, Charu Aggarwal, Springer International Publishing AG (2018).

FIGS. 2c and 2d are examples of pixel maps (the intensity of the pixel is related to its value) of “FastGraph” type descriptor for an atom in different crystallographic structures: centred cubic (CC), face centred cubic (FCC), hexagonal close-packed (HCP) and cubic diamonds (diam), showing the visual differences which can be exploited for this phase of classification.

Additional details regarding this descriptor are given later in the present description; for example the 1^{st} line describes the atomic environment of a central atom, the 2^{nd} the environment of the 1^{st} atom closer to the central atom and so on and the k^{th} line the environment of the k^{th} atom closer to the central atom.

Within the scope of the present invention, other descriptors could be used, for example of the type which are numerically very heavy (and involve the implementation of very strong computing resources) but which are accurate, or of the type of those which are inaccurate, but more efficient and faster from the numerical point of view.

For example, it is possible to use the descriptor SO4 (bSO4) described in the article by Gorayeva et al. which is already commented above, this descriptor being numerically very heavy but accurate. It can be used with a classifier implementing a dense neural network (NN) in order to identify the crystallographic structure of each atom under conditions close to those encountered in TAP-type experiments. A solution according to the present invention, combining a “FastGraph” type descriptor with a convolutional neural network (CNN), is much faster and offers the same accuracy as the SO4 descriptor.

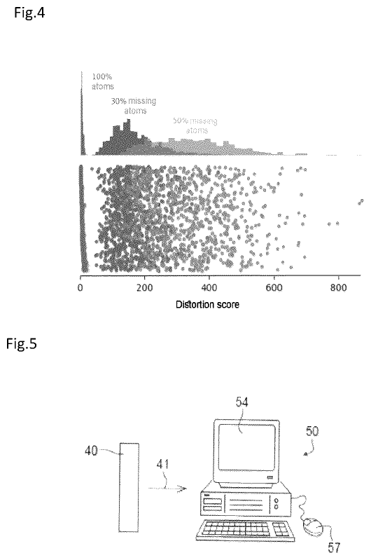

FIG. 4 illustrates the accuracy obtained with a method according to the invention with the four crystallographic structures (mentioned above) most common in materials science. An in silico database was created with these 4 types of crystallographic structures: CC (Fe), FCC (Cu), HCP (Fe at very high pressure) and diamond (Si). Preferably, these structures are in a highly disturbed state, with a high temperature, up to ⅔ of the melting temperature. Atoms were gradually removed, up to 50%, which is a situation frequently encountered in TAP-type experiments. It was then possible to notice that:
the conventional methods, with unimodal distribution or not, such as Ovito PTM or CNA, fail, even with a small fraction of missing atoms;
the “FastGraph” method with a convolutional neural network (CNN) gives 100% accuracy, even with 50% missing atoms, at the same level as the BSO(4) descriptor, which is about 5000 times heavier, combined with a dense neural network.

In this same step, a statistical pre-analysis can be carried out for the experimental data 4_{d}, to take into account the multi-modal nature of the data. The sub-adjacent statistical distribution of data in the descriptor space being multimodal, the reference data is distributed into several groups. Each group, for example, consists of data that can be described with a single Gaussian distribution. A concrete example is the distribution of the atomic positions measured in TAP which have two systematic errors inherent to the TAP technique itself: an error associated with the normal direction of detection Z (also direction of evaporation) and another error (about 10 times larger) associated with the lateral directions X and Y. This step can also allow obtaining the values of these systematic errors. The same analysis can also be carried out for the reference samples 2_{d}.

This analysis can be done using a Gaussian Mixture type method (as described in the works C. M. Bishop: Mixture density networks (1994) or M. P. Deisenroth et al. Mathematics for machine learning, Cambridge University Press, (2020)).

Depending on this pre-analysis, the experimental confidence score, discussed below (step 3) will have one or more dimensions, depending on the number of groups.

In a third step (S3), a step of calculating the experimental confidence score (based on “learning” the statistical distribution of the data, of step 2 (2_{d} and 4_{d})) is carried out. This method is for example a machine learning or deep learning method, it can for example be a Mahalanobis type statistical distance calculation (P. C. Mahalanobis, Proceedings of the National Institute of Sciences of India, 2, 49-55 (1936)), or else the MCD method (described for example in M. Hubert et al., Minimum covariance determinant and extension, 10, e1421, WIRES Comp. Stat. (2018) or in P. J. Rousseeuw et al. A fast algorithm for the Minimum Covariance Determinant estimator, Technometrics 41, 212-223 (1999)) or Mahalanobis (P. C. Mahalanobis, Proceedings of the National Institute of Sciences of India, 2, 49-55 (1936)) if the multimodal aspect of the experiment is well known and usable. Alternatively, it is possible to implement an SVM type technique (“Support Vector Machine”, see for example Vapnik, V. N. The Nature of Statistical Learning Theory, Speinger-Verlag, New York, 1998) or a neural network (see in particular: C. M. Bishop: Mixture density networks (1994) or M. P. Deisenroth et al. Mathematics for machine learning, Cambridge University Press, (2020)).

In order to best process experimental data with multimodal distributions, a highly nonlinear artificial intelligence model is preferably used, such as a neural network or of the SVM (“support vector machine”) type.

This step therefore allows associating an experimental confidence score with each atom. The dimension of this score can be defined by the statistical pre-analysis mentioned above, relative to the number of groups identified in step S2, at the end of the pre-analysis of the statistical distribution of descriptors using for example the “Gaussian Mixture” method. The amplitude of the experimental distortion confidence score, according to each dimension, is calculated relative to the corresponding group.

In a fourth step (S4), an anomaly detection (at the scale of atoms or domains which, potentially, correspond to the defects) is carried out. Depending on the experimental confidence score established during the previous step, using a classification algorithm, it is possible to “label”, for example for the atoms from TAP data, the “normal” and “unusual” cases. It is therefore possible to stratify the scores obtained relative to a threshold which will ultimately allow detecting the differences between the noise and the real clusters. The implemented classification algorithm can for example be of the type:
DBScan; see for example M. Ester et al. A density-based algorithm for discovering clusters in large spatial databases with noise, Proceedings of the 2nd International Conference on Knowledge Discovery and Data mining, 1, 226-231 (1996);
or neural network; see on this subject, in addition to the already mentioned references, for example Neural Networks and Deep Learning: A Textbook, Charu Aggarwal, Springer International Publishing AG (2018); or A. P. Dempster et al.: Maximum likelihood from incomplete data via the EM algorithm, Journal of the Royal Statistical Society: Series B (Methodological), 39, 1-22 (1977); or G. Heinz et al.: Exploring Relationships in Body Dimensions. Journal of Statistics Education, 11, 2 (2003); or K. P. Murphy: Machine learning: a probabilistic perspective., MIT press (2012))
or SVM “Support Vector Machine”, Vapnik, V. N. The Nature of Statistical Learning Theory (Springer-Verlag, New York, 1998)., or MCD (see references which are already mentioned for this method) or any other “clustering” method.

In a fifth step (S5) it is possible to carry out a distribution or a grouping of the atoms detected by class of defects. For this, a clustering method by machine learning or deep learning is implemented: DBSCAN (see reference which is already mentioned above about this method), or a “Gaussian Mixtures” or neural network type method (see the references which are already mentioned above on this subject).

It is then possible, during a sixth step (S6), depending on the morphology and/or the geometry of the clusters identified in the previous steps, to carry out a physical interpretation: for example, the 2D type clusters can be interpreted such as loops or dislocation lines, the 3D type clusters such as precipitates or cavities.

As already indicated above, the experimental data, processed by a method according to the invention, can be obtained other than by the Tomographic Atom Probe (TAP) technique. They can be obtained by Transmission Electron Microscopy (TEM) or by X-ray diffraction (XRD) for example from Synchrotron Radiation (RS). Using the TAP technique, it is possible to access the positions of the atoms, whereas, in the TEM or XRD techniques:
work is on images, the atoms being replaced by pixels (data obtained on TEM or XRD detectors);
the atomic descriptors are replaced by image descriptors.

It is therefore possible to carry out a detection and/or a morphological characterisation of defects such as the irradiation loops, or the detection and the identification of clusters of segregated elements within a homogeneous solid solution.

For each of the implemented techniques, the digital data preferably takes into account:
specific characteristics of this analysis technique, for example spatial and chemical uncertainties;
and/or the different behaviours of the atoms during the implementation of the considered technique, such as the crystallographic direction and/or the presence of another phase (clusters, or defects . . . ) within the analysed sample.

The data implemented during each of the steps of a method according to the invention can be processed by a system such as a processing unit or a calculator (for example: a computer, or a microcomputer, or a server).

A more detailed description of the “FastGraph” descriptor will now be given. One approach can be based on the representation of the local neighbourhood of a central atom j in a 2D image which is invariant in rotation and in permutation. It is the visual representation by a matrix of pixels of a dense, non-directional graph, with the nodes or vertices of the graph being, or corresponding to, the very atoms of the atomic environment of a central atom and with edges with weight weighted by the interatomic distances. Consider the set v (j) of neighbours of the atom j with a cut-off radius (for example as defined above) rₒᵤₜ=(<<v(j)={i|rᵢⱼ≤r_{cut},i≠j}>>). The cardinal n(j) of this set (n(j)=|v(j)|) is the number of neighbours of the atom j. it is noted αj:v(j)→ {1, . . . , n(j)} the bijective relation which transforms the elements of v(j) into a 1 sequence of integers from 1 to n(j). The relation αⱼ assigns the number “1” to the atom which is closest to the atom j, the number 2 to the second closest neighbour and so on until n(j), which is the n(j)^{th} closest neighbour of the atom j. It is noted Gⱼ the graph which has the n(v)+1 nodes denoted from 0 to n(v) (the node 0 of the graph is the atom itself) and whose edges represent the connections between the atoms. It is noted r_{j:0k} the distance, in the graph Gⱼ from the central node 0 of Gⱼ to the closest neighbour k of the node 0. Similarly, it is possible to measure the distance r_{j:lk} which is the distance between the l^{th} neighbour of the node 0 and the kth neighbour, in Gⱼ, of the lth neighbour of the node 0 in the same graph Gⱼ.

In the set v (j), n_{G}−1 atoms (the first n_{G}−1 neighbours) are selected. Preferably, n_{G}−1 are selected between, on the one hand, 35 or 31 and, on the other hand, 15 or 10 (optimal value or range); in general, this number is selected lower than the average number of n(j) (in the database).

First, it is possible to treat the case of (n_{G}−1)≤n(j) for any atom j. The graph Gj is in the form of an n_{G}×N_{G} matrix. The 1^{st} row contains n_{G} pixels, each of which having a value representing, or related to, the inverse of r_{j:0k} (with k ranging from 1 to n_{G}). The lth line (1<l≤n_{G}) of the matrix Mⱼ concerns the neighbour of order (l−1) of the node 0 of the graph Gⱼ, again with n_{G} pixels which are inversely proportional to r_{j:(l-1)k} (with k from 1 to n_{G}). The case (n_{G}−1)>n(j) can be treated by assigning zero values to the elements of the matrix M.

An implementation with a single chemical element has been described above. A multi-element version, adapted to the alloys or molecules can be deduced from what is explained above: the intensity of each pixel of the matrix can be modified proportionally with a given weight factor for each chemical element. The usefulness of this description appears in FIGS. 2a-2d, which have already been commented on above. The unique design of this descriptor enables the easy implementation of a convolutional neural network (CNN) which can classify the “FastGraph” descriptor of each atom. The combination of this descriptor with a CNN allows an efficient and rapid processing of experimental data.

The multimodal nature of experimental data is explained below, in particular in the case of experiments carried out by tomographic atom probe (TAP).

In this type of analysis, the material is examined and prepared in the form of a very fine tip evaporated under the action of an electric field; it is the best characterisation technique to perform measurements providing information from a 3D image at the atomic scale and/or the chemical composition of the material, with a spatial resolution at the atomic scale. In principle, this technique would allow providing the position of each atom in a structure with a sufficient accuracy to determine the atomic arrangement in the material.

However, as with any microscopy technique, there are many obstacles to overcome in order to achieve the optimal spatial resolution. The accurate 3D images are impaired by the quantum nature of the atom detection, which implies that approximately every two seconds an atom is missing from the ultimately detected structures. In addition, the results of the experiments generally contain geometric reconstruction artifacts related to the shape of the TAP tip (sample for the TAP analysis, sharply cut). Consequently, in the best TAP experiments it is possible to detect the 3D arrangements of the atoms with a near-atomic spatial resolution, which is 3 Å in the lateral detection direction and 1 Å deep in the long direction of the TAP tip.

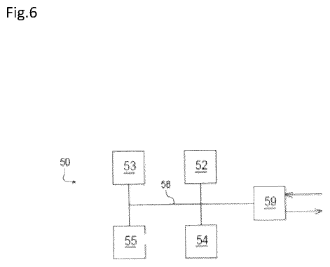

An example of such a system is shown in FIGS. 5 and 6. It includes for example means 50, for example a computer or a calculator or microcomputer, to which a sensor 40 transmits measurement data via a link 41. For example, in the case of the implementation of a tomographic atom probe technique, the sensor 40 is an ion detector, which allows measuring the time of flight of the ions and their positions; in the case of an analysis by transmission electron microscopy this sensor 40 is a camera; The same applies in the case of an X-ray analysis, for example from Synchrotron radiation. According to one embodiment, the means 50 include (FIG. 6) a microprocessor 52, a set of RAM memories 53 (for storing data), a ROM memory 55 (for storing program instructions). Possibly, means, for example a data acquisition card 59, transform the analog data provided by one or more sensors into digital data and put this data in the format required by the means 50.

These various elements are connected to a bus 58.

Peripheral devices (screen or display means 54, mouse 57) allow an interactive dialogue with a user. In particular, the display means (screen) 54 allow providing a user with a visual indication.

In the means 50, the data or instructions are loaded to implement a data processing according to the invention, and in particular to perform the training of one or more model(s) and/or to carry out possible prior processing of the data.

These data or instructions for training a model and/or for carrying out a possible prior processing of the data and/or the experimental measurement data(s), the reference structure data and/or the descriptor space (or the data to generate it) and/or one or more descriptor function(s) and/or to perform the calculation of an experimental confidence score and/or a classification (in particular the data relating to one or more machine learning method(s)) and/or any other data to implement the invention, may be in a memory area of the means 50, in which they may have been transferred for example from any medium capable of being read by a microcomputer or a computer (for example: USB key, hard disk, ROM read-only memory, DRAM dynamic random access memory or any other type of RAM memory, compact optical disk, magnetic or optical storage element). The invention allows:
the processing and the interpretation of experimental data with an atomistic resolution and the identification and/or the classification of defects;
accelerating the data analysis;
an exploitation of data in a reproducible, non-biased manner and without human interpretation error.

The invention relates to a method and a device adapted for the analysis of experimental data as well as their interpretation based on the characteristics specific to each type of experiment. The interpretation of data from experiments at the atomic scale has never before been the subject of an analysis in an abstract descriptor space.

1. A computer-implemented method for processing experimental data of a solid to be characterized, including atoms and including one or more defects, said experimental data coming from at least one sensor and having a multimodal distribution, the method comprising:
representing, in a descriptor space of dimension K, comprised between 10 and 10⁸, one or more reference solid(s) and said data;
calculating, for at least one portion of the atoms of the solid to be characterized, an experimental confidence score in the descriptor space, relative to the atoms of said reference solid; and
classifying the atoms of the structure depending on the experimental confidence score.

2. The method according to claim 1, wherein the experimental is obtained by a Tomographic Atom Probe (TAP) technique, by Transmission Electron Microscopy (TEM), or by X-ray diffraction.

3. The method according to claim 1, further comprising a prior step of forming the descriptor space and/or one or more descriptor function(s), depending at least on distances between the atoms and/or angles between directions connecting each atom of the solid or the sample which is studied to different neighbours in the network of the solid to be characterized.

4. The method according to claim 1, wherein the representation, in the descriptor space of dimension K, preserves symmetry (symmetries) and the chemical nature of the atomic structure(s) resulting from the experiment and/or used for reference.

5. The method according to claim 1, wherein the representation step is performed using a descriptor which implements, for each atom j, a graph Gj whose nodes are neighbours, more or less close, to the atom j, the graph then being pixelated in a form of a matrix Mⱼ.

6. The method according to claim 5, wherein the graph is a dense, non-directional graph, the nodes or vertices of the graph corresponding to the atoms of the atomic environment of a central atom, and with edges with weight weighted by interatomic distances.

7. The method according to claim 5, the lth line (1<l≤n_{G}) of the matrix Mⱼ concerning the neighbour of order (l−1) of the node 0 of the graph (Gⱼ).

8. The method according to claim 1, further comprising a preliminary step of selecting a cut-off radius, which defines an environment of one or more atom(s) or of each atom j, the environment including all atoms present in a vicinity of the atom j or of each atom j and which are included in the cut-off radius.

9. The method according to claim 1, further comprising learning a method for calculating a statistical distance of said experimental confidence score.

10. The method according to claim 9, wherein the step of learning the method for calculating an experimental confidence score implements a machine learning or a deep learning method, an anomaly detection or a novelty detection method, including one or more of a statistical distance calculation, an MCD, a Mahalanobis type method, a physical statistical distance calculation, an SVM type technique, or a neural network.

11. The method according to claim 1, wherein the classification of the atoms implements a classification algorithm of the DBScan, a neural network, an SVM, a MCD, or other clustering method type.

12. The method according to claim 1, further comprising distributing or grouping the atoms detected by class of defects, by a machine learning or deep learning type method or a clustering and classification method.

13. The method according to claim 1, further comprising distributing or grouping the atoms detected by class of defects by a convolutional neural network type method.

14. A device for processing experimental data from solids to be characterized, including atoms and including one or more defects, said data having a multimodal distribution, the device comprising:
means configured to represent, in a descriptor space of dimension K comprised between 10 and 10⁸, at least one reference solid and said data,
means configured to calculate an experimental confidence score, in the descriptor space, for at least one portion of the atoms of said solid to be characterized, relative to the atoms of said reference solid; and
means configured to classify atoms of a solid depending on said experimental confidence score.

15. The device according to claim 14, the device being connected to a detector, being a detector of a Tomographic Atom Probe (TAP) system, an X-ray detector associated with a Transmission Electron Microscopy (TEM) system, or an X-ray diffraction system.

16. The device according to claim 14, further comprising means configured to form or calculate the descriptor space from experimental data of at least one reference sample depending on at least distances between the atoms and angles between directions connecting the atoms of the solid.

17. The device according to claim 14, further comprising means configured to implement a representation step using a descriptor for which, for each atom j, a graph Gj whose nodes are neighbours, more or less close, to the atom j, the graph then being pixelated in the form of a matrix.

18. The device according to claim 17, wherein the graph is a dense, non-directional graph, the nodes or vertices of the graph corresponding to the atoms of the atomic environment of a central atom, with edges with weight weighted by the interatomic distances.

19. The device according to claim 17, the lth line (1<l≤n_{G}) of the matrix Mⱼ concerning the neighbour of order (l−1) of the node 0 of the graph (Gⱼ).

20. The device according to claim 14, including comprising means configured to implement a step of processing or preprocessing and/or preparing experimental data.

21. The device according to claim 14, further comprising means configured to implement a step of learning a method for calculating, for example a statistical distance of said experimental confidence score.

22. The device according to claim 14, further comprising means configured to implement a machine learning or deep learning method or an anomaly detection or novelty detection method, including one or more of a statistical distance calculation, an MCD, a Mahalanobis type method, a physical statistical distance calculation, an SVM type technique, or a neural network.

23. The device according to claim 14, further comprising means configured to implement a machine learning or deep learning method or an anomaly detection or novelty detection method, by a convolutional neural network.

24. The device according to claim 14, further comprising means configured to implement a classification algorithm of the DBScan, neural network, an SVM, a MCD, or other clustering method type.

25. The device according to claim 14, further comprising means configured to perform a distribution or a grouping of the atoms detected by class of defects, by a machine learning or deep learning type method, or a clustering and classification method.

26. The device according to claim 14, further comprising means for selecting a cut-off radius, which defines the environment of one or more atom(s) j or of each atom j, this environment including all atoms present in the vicinity of the atom j or of each atom j and which are included in the cut-off radius.
